(19) United States
(12) Patent Application Publication
(10) Pub. No.: US 2026/0360680 A1
(43) Pub. Date: Nov. 12, 2026

(54) ANOMALY DETECTION IN AN ENVIRONMENT USING SINGLE PARTICLE AEROSOL MASS SPECTRA

(71) Applicant: Zeteo Tech, Inc., Sykesville, MD (US)

(72) Inventors: Ross Kliegman, Sykesville, MD (US); Michael McLoughlin, Sykesville, MD (US)

(73) Assignee: Zeteo Tech, Inc., Sykesville, MD (US)

(21) Appl. No.: 19/122,460

(22) PCT Filed: Oct. 20, 2023
(86) PCT No.: PCT/US2023/035592

§ 371 Date: Apr. 18, 2025

Related U.S. Application Data
(60) Provisional application No. 63/417,953, filed on Oct. 20, 2022.
(60) Provisional application No. 63/544,859, filed on Oct. 19, 2023.

(51) Int. Cl. H01J 49/04; G01N 27/623

(52) U.S. Cl. CPC H01J 49/0445; G01N 27/623

(57) ABSTRACT

Methods and systems to detect an anomaly caused by unknown aerosol hazardous particles in an environment using single particle mass spectra of environmental aerosol samples. An autoencoder is trained using a dataset of single particle mass spectra to diagnose an anomaly in the environment. To test the autoencoder's ability to predict known hazardous substances as anomalies, a reference mass spectra data set is generated using simulated composite mass spectra of hazardous analyte particles in the background environment in silica at different analyte concentrations.

### CROSS-REFERENCE TO RELATED APPLICATIONS

This Patent Application is related to and claims the benefit of U.S. Provisional Pat. Appl. No. 63/417,953, filed Oct. 20, 2022, and titled “ANOMALY DETECTION IN AN ENVIRONMENT USING SINGLE PARTICLE AEROSOL MASS SPECTRA,” and U.S. Provisional Pat. Appl. No. 63/544,859, filed Oct. 19, 2023, and titled “ANOMALY DETECTION IN AN ENVIRONMENT USING SINGLE PARTICLE AEROSOL MASS SPECTRA, the disclosures of which are incorporated by reference herein in each of their entireties.

### FEDERALLY SPONSORED RESEARCH AND DEVELOPMENT

None.

### TECHNICAL FIELD

This disclosure relates to methods and systems to detect an anomaly caused by unknown biological containing particles in an environment using single particle mass spectra of environmental aerosol samples. More particularly, but not by way of limitation, the present disclosure relates to methods and systems for detecting an anomaly caused by known and unknown airborne biological and chemical containing particles by training an autoencoder to analyze single particle mass spectra of aerosol samples from ambient air. For testing the autoencoder on anomalies caused by known biological substances, reference mass spectra data sets are generated in silico, that is, computationally or using computer simulation, using simulated composite mass spectra of hazardous or threat agent particles in the background environment at different hazardous substance (analyte) concentrations.

### BACKGROUND

The threat from aerosolized biological and chemical hazardous substances or other hazardous substances remains a key concern of the U.S. Government because of the potentially dire consequences to life and property that may result from such an event. Two prime threat or hazard scenarios of particular concern are: (1) release of an agent or hazardous substance inside an enclosed structure (e.g., office building, airport, mass transit facility) where HVAC systems could effectively distribute the agents through the entire structure and, (2) wide area release of an agent or hazardous substance across an inhabited area such as a town or city. Exposure to the released aerosolized hazardous substance could lead to mass casualties. In a wide area release, it is extremely difficult to protect citizens from the initial exposure without timely information about the type of contaminant, quantity, and location of the contaminant. Methods and devices to identify the composition of the threat agents or hazardous substances, or other related biological and chemical substances in real time are required to take quick remedial action. A sample of an analyte aerosol in air may be captured using suitable means such as a filter designed to capture respirable particles, sampling bag, and other similar enclosures. The particles may also derive from a liquid sample obtained from a wet-wall cyclone or similar device that has been subsequently re-aerosolized. An example of a wet-wall cyclone is the SpinCon II (Innovaprep, Drexel, MO). The particles in these aerosols could include, but are not limited to anthrax, Ebola virus, ricin, and botulinum toxin. All of these collection methods require additional processing to extract biological particles for analysis, resulting in delays of hours or days to detect and identify a hazardous aerosol.

Solutions to detect and analyze aerosol analytes, such as biological agents and hazardous substances are available but do not permit quick or real-time analysis. One solution employs microfluidic techniques to clean-up the sample and concentrate the biological analyte. For example, specific antibodies may be employed to concentrate and purify the biological analyte. This target-specific solution provides reasonable results if sufficient time is allowed for clean-up and concentration of the analyte. Another solution is target-specific and works only for bacterial analytes at the expense of analyzing viruses, toxins, particulate chemicals or other non-culturable components of the aerosol. This method requires a sample, for example from a patient, to be applied to a bacterial culture plate and incubated for 8 to 24 hours. After the bacterial colonies have grown, individual amplified and purified colonies are collected and measured by whole cell matrix-assisted laser desorption/ionization time-of-flight mass spectrometry (“MALDI TOF-MS”). Numerous studies have examined the accuracy of this technique and have found >99% accurate identification for clinical bacterial analytes. Two commercial systems for rapid clinical bacterial identification have been developed, namely, the Bruker Biotyper (marketed by Becton Dickinson) and the Shimadzu Vitek MS (marketed by bioMérieux).

In conventional MALDI mass spectrometry, the sample to be analyzed is first mixed with a MALDI matrix and then placed on the probe tip within the mass spectrometer vacuum chamber. The MALDI matrix resonantly absorbs ultraviolet (337 nm) laser light pulses and simultaneously desorbs the analyte into the gas phase as molecular ions. In aerosol MALDI mass spectrometry, the aerosolized analyte particles are preferably coated with the MALDI matrix on-the-fly. For example, commonly owned patent application U.S. application Ser. No. 15/755,063 discloses a method for coating aerosol particles including aerosolizing a coating material (MALDI matrix) to form a first aerosol including liquid particles, providing a sampled aerosol containing analyte particles to form a second aerosol, providing an acoustic coater to receive the first aerosol (MALDI matrix) and the second aerosol, and providing an acoustic field to the acoustic coater to urge the first aerosol (MALDI matrix) to impinge upon particles of the second aerosol on-the-fly to form coated aerosol particles including a coating of first aerosol on the second aerosol particles. Other aerosol particle coating methods may also be used.

Example MALDI matrix chemicals may include at least one of 2,5-dihydroxybenzoic acid, alpha-cyano-4-hydroxycinnamic acid, 3,5-dimethoxy-4-hydroxycinnamic acid, 2-mercapto-4,5-dialkylheteroarene, 1,8-dihydroxyanthracen-9 (10H)-one, 3-methoxy-4-hydroxycinnamic acid, 2,4,6-trihydroxyacetophenone, 2-(4-hydroxyphenylazo)-benzoic acid, trans-3-indoleacrylic acid, 4-hydroxy-3-methoxybenzoic acid, 6-aza-2-thiothymine, 2-amino-4-methyl-5-nitropyridine, 4-nitroaniline, 1,5-diaminonaphthalene, 5-fluorosalicylic acid, 5-chlorosalicylic acid, 5-bromosalicylic acid, 5-iodosalicylic acid, 5-methylsalicylic acid, 5-aminosalicylic acid, and 1,8-diaminonaphthalene. Additionally, example MALDI matrix solution may include at least one of acetonitrile, water, ethanol, methanol, propanol, acetone, chloroform, isopropyl alcohol, tetrahydrofuran, toluene, hydrochloric acid, trifluoracetic acid, formic acid, and acetic acid.

Once ions are created in the MALDI process, they may be analyzed in a time-of-flight mass spectrometer (“TOF-MS”). In a TOF-MS, data is recorded as a time series of voltages as ions strike the detector. Flight times of the ions are converted to mass as described below. A linear TOF-MS is schematically shown in FIGS. 1A and 1s typically used for conventional MALDI mass spectrometer systems. Ions are formed in a short source region of length (“s”), generally defined by a backing plate and an extraction grid. A voltage (“V”) placed on the backing plate imposes an electric field (“E”) across the source region, where E=V/s. This electric field accelerates these ions to the same kinetic energy (“U”), which may be defined by U=mv²/2=ze(V)=ze (Es), where m is the mass of the ions, v is the ion velocity, e is the charge on an electron, and z is the number of charges on the ions. The charge, z, is equal to 1 for most ions, though higher values are occasionally seen, especially for larger ions. As the ions pass through an extraction grid, ion velocities depend inversely on the square root of their mass-to-charge ratio as:

$$v = {( \frac{2 z E s}{( \frac{m}{e} )} )}^{1 / 2}$$

The ions pass through a much longer drift region of length (“D”) where they separate in time such that the mass spectrum at various time-of-flight t is generated as:

$$\frac{m}{z} = 2 e E {s ( \frac{t}{D} )}^{2} = {kt}^{2}$$

Typically, the relationship between the mass-to-charge ratio (m/z) and the time-of-flight t is calibrated using compounds of well-known mass m in a known charge state z to determine the lumped quantity k to yield the relationship between mass and time in a TOF-MS.

For higher resolution, the reflectron TOF mass spectrometer mode shown in FIG. 1B may be used to enhance mass resolution. In this configuration, new ion optics elements are introduced to provide a potential “hill” that turns the ions around. The reflectron includes a series of plates 101, which are installed in the mass spectrometer and wired in serial fashion such that a potential gradient is formed. The end of the reflectron closest to source 102 is held at ground potential and each successive plate steps the voltage up to the ultimate voltage (V+δ), which is slightly higher than the source voltage (V). Ions are formed as described in the linear TOF mode, but instead of being detected in a linear fashion by linear detector 103, they pass into the reflectron where they climb the potential energy gradient, stop, and then turn around and accelerate to the reflectron detector 104. The reflectron mode helps to substantially diminish the spread of flight times of the ions with the same mass caused by spread in kinetic energy of these ions at the exit from the ion source.

The reflectron configuration may be turned ON/OFF and operated as a linear TOF instrument. If the voltage on the reflectron element is turned OFF, the ions travel through the grounded reflectron elements and strike detector 103 placed at the end of the spectrometer. In this case, the mass spectrometer can rapidly switch between the high mass range and sensitivity provided by the linear TOF mode and the high-resolution provided by a reflectron TOF.

Analysis of biothreats using conventional MALDI TOF MS has been reported by the Johns Hopkins University's Applied Physics Laboratory (JHU-APL) using a top-down proteomics approach. A biothreat agent is exposed to a laser pulse which causes reproducible whole molecular fragments to be created. The exact masses of these pieces or fragments are measured by the TOF-MS to generate a plot of intensity as a function of mass and provide a mass spectrum. As an example of the whole-cell or top-down approach, FIG. 2 shows the mass spectrum of Bacillus globigii (“Bg”) in the spore state. The current nomenclature of this organism is Bacillus atrophaeus and there are many strains and related materials from laboratories around the world. Bg has historically been used as a non-infective simulant for Bacillus anthracis, which is a Tier 1 CDC agent and generally considered as the most likely agent or biological hazardous substance of choice for bioterrorists.

A Bacillus atrophaeus sample was mixed with a MALDI matrix in a solvent, deposited on a surface, allowed to dry, inserted into the TOF-MS and analyzed. The resulting spectra characteristic of Bacillus atrophaeus spores is shown in FIG. 2. The collective features in the spectrum represent the signature of Bacillus atrophaeus and the genetic structure of this threat organism, are reproducible, and are characteristic of Bacillus atrophaeus. These features are not impacted by growth conditions or media, preparation methods or environmental contaminants. The numbers annotated in FIG. 2 represent the m/z (Daltons) values of the peaks as determined by time of arrival of the ion packet at the detector and then subsequently converted to mass by calibration of the instrument with substances of known mass, as previously described. The spectrum also shows the molecular identities of many of the main peaks as annotations, using information from detailed mass spectrometry with the known gene sequence of the organism, and published reports on the occurrence and role of small acid soluble proteins (“SASP”) in the spore coat of Bacillus species. Differences in the genetic structure of the members of the different Bacillus species result in differences in the masses of the SASP peaks and provides a mechanism for differentiation by MALDI mass spectrometry. It has been reported that strong acid treatment of spores can rapidly extract the small acid soluble proteins that have become the characteristic biomarkers of Bacillus spore identification.

These systems provide excellent diagnostic results relative to the 16s RNA “gold standard.” However, to achieve these high-confidence clinical results, either a culturing or an extraction step, or both, is needed to purify the sample. Therefore, the time from sampling to identification of the bio-analyte is generally twelve hours to a day or more. While such delays are often tolerable in clinical laboratories, they are often unacceptable for other applications such as biodefense, where real-time identification of bio analytes is needed. Biodefense, as well as point-of-care healthcare applications, requires the ability to simultaneously identify in real-time not only bacteria, but also fungi, viruses and large bioorganic molecules (e.g., proteins, peptides and lipids) including biotoxins. Further, decreasing analysis time for clinical applications could improve quality of care and outcomes by enabling more timely treatment and identification of the best course of treatment (for example distinguishing between viral and bacterial infection) and evaluation of the effectiveness of the course of treatment.

For aerosol analysis, commonly owned patent application Intl. Appl. No. PCT/US20/40023 discloses an example aerosol TOF-MS and is incorporated by reference herein in its entirety. In example system 300 (FIG. 3), aerosol particles, for example, particles including biological matter in air, are routed to a suitable inlet element 301 that removes debris and materials from the particles, at rates of about 1000's of particles per second, and flow into an aerosol beam generator 302 that collimates the particles into a narrow beam of single particles. Prior to entering the beam generator 302, the aerosol may pass into a MALDI matrix processing subsystem 313 where particles are coated with MALDI matrix and processed for analysis. The particles then pass through the aerosol beam generator in which the pressure is dropped from atmospheric pressure to the base pressure on the mass spectrometer (about 10⁻⁵ to 10⁻⁶ Torr) using differential pumping. The beam generator utilizes differential pumping to reduce the pressure to a level that is compatible with the high vacuum in chamber 304. The particles may be indexed using a continuous laser from laser generator 303 (e.g., commercially available laser scattering devices that include but are not limited to, IBAC and Polaron systems). In addition, the continuous laser may be used to determine particle size, fluorescence (autofluorescence) and polarization (particle shape) and identify particles of particular interest. The particles then travel into vacuum chamber 304 through a series of focusing lenses. This chamber may house an advanced time-of-flight mass spectrometer (TOF-MS) 306 and optionally, light collection optical components 307.

The IBAC system uses UV laser induced fluorescence to measure aerosol particle data. Laser Induced Fluorescence (“LIF”) excites a particle or sample with a laser (the continuous laser as above may be used) and the emitted fluorescence may be detected an analyzed using a suitable photo detector including a photomultiplier tube (“PMT”) detector to distinguish between biological particles and non-biological particles. Using fluorescence to determine biological aerosol particle concentrations and size distributions measured with an ultraviolet aerodynamic particle sizer is well known in the art. A Polaron uses polarized elastic light scattering produced from laser-excited particles (the continuous laser as disclosed above may be used) to classify aerosol particles based on particle shape and size.

As each indexed particle enters the center of chamber 304, it is struck with a high-power laser pulse from laser generator 308. Aerosol mass spectrometry requires the ionization laser 308 to fire when the aerosol particle enters the region illuminated by the laser (typically <150 microns in diameter). Because the pulse ionization laser 308 fires a pulse that is less than 5 ns (nanosecond) in duration, advanced knowledge is required to predict when a particle will enter the ionization region and trigger the laser 308. Multiple lasers may be used to measure and track particles to predict the time at which a particle will enter the view of the laser. In the example system, at least one of the laser from generator 303 and the laser from laser generator 312 may be used to index and detect particles as they leave the beam generator 302. Because both laser beams 308 and 312 are closely aligned, a single trigger laser 312 is sufficient to predict the path of a single aerosol particle and trigger the pulse ionization laser 308, greatly reducing the complexity of the particle timing hardware.

Pulse ionization laser 308 may also be triggered using the laser from generator 303. Laser 308 may be triggered only when at least one of particle size, shape, and fluorescence meets or exceeds a predetermined threshold value for that property. When monitoring the composition of aerosol particles in ambient air at periodic intervals, the selective triggering of laser 308 in this manner, and subsequent examination of ionized fragments of each particle and analysis of the data collected may be controlled (or tuned) to avoid collection of superfluous data and improve data management. The timing (or trigger) laser 312 may also be used to measure optical properties of the particle (e.g., size, shape or fluorescence). These measurements can be used to select particles to be ionized, and data can be combined with mass spectral measurements and other optical information obtained during ionization for analysis in data analysis system 310 using data fusion methods. The intensity of laser pulse from generator 308 may be tuned such that the particle is deconstructed to generate ions from the constituent biochemical components. That is, the laser vaporizes and ionizes at least some of the analyte molecules, thus generating ions with specific mass to charge ratios (m/z). These large, informative ions are accelerated into the TOF-MS 306 where they are analyzed.

Additionally, when the analyte particles absorb sufficient light energy from a laser beam, they emit characteristic photons as they transition from a high-energy state to a lower energy state. Light emissions could also be associated with transitions between vibrational states. The interaction of the high-power laser pulse generated by generator 308 with the particles may also induce transient optical signatures such as high-order fluorescence, laser-induced breakdown spectroscopy (“LIBS”), Raman spectra and infrared spectra. Chamber 304 may also include light collection optical components 307. Unique spectral data associated with each particle and generated using the TOF-MS and optical sensors 309, and particle specific data (e.g., particle size, shape, fluorescence) from laser devices 303 and 312 may undergo data processing including data fusion in data analysis system 310 to generate compiled spectral data associated with each particle. The compiled spectral data may be compared with a training data set including a knowledge base of known biological matter spectra to predict composition. System 310 may be in data communication with machine learning engine 311 to allow for updating the training data set knowledge based and improving the prediction of composition over time. The pressure in chamber 304 is reduced to at least 10⁻⁵ torr using vacuum pump 305. In example system 300, the travel time (or residence time) of a particle from beam generator 302 to being hit with laser 308 is less than about 1 s.

The creation of aerosol single particle MALDI mass spectrometry signatures is fundamentally different than that obtained from conventional MALDI mass spectroscopic methods or for any other mass spectrometry method that interrogates a solid bulk sample. Conventional MALDI mass spectrometry extracts ions from a bulk sample which includes a large number of particles, typically in the 1000's. These particles include possible pathogenic organisms of interest, including, but not limited to, bacteria and viruses, their constituent materials characteristic of the pathogens, including, but not limited to, proteins, peptides, and lipids, reagents (e.g., MALDI matrix) and contaminants (e.g., environmental material, material and byproducts associated with humans such as, for example, sputum from a breath sample or cough sample. These particles of interest are dispersed throughout the sample and are mixed with other particles such as environmental contaminants. The spatial distribution of particles in the bulk sample causes a distribution of the distances and times of flight (to the detector) of the ions created from the sample when the sample is impacted by an ionization laser. The ion spread can be somewhat reduced by design of the ion source region, for example, by employing methods such as delayed extraction and two-stage extraction.

Additionally, to reduce noise and improve the signal to noise ratio, multiple laser shots are typically performed and the spectra from each individual shot are averaged. While averaging improves the signal-to-noise of peaks by reducing noise associated with the spectrometer, the variability due to the inhomogeneous nature of the sample is not reduced. Attempts to use individual measurements and average or mean spectra for denoising and alignment of characteristic peaks have shown that using the mean spectra provides better results, because each bulk sample includes a variety of particles of different make-up, and a single measurement from these particles generates ions related to these disparate particles. As such, it is not advantageous to deconvolve mass signal components related to individual particles during conventional mass spectrometry of bulk samples. A similar challenge is seen during deconvolution of the signal components related to individual particles from an aerosolized sample.

Commonly owned patent application U.S. patent application Ser. No. 17/507,755 discloses a system to identify the composition of aerosolized particles and is incorporated by reference herein in its entirety. The disclosed system includes an aerosol beam generator to generate a beam of single particles, a continuous timing laser generator to generate a timing laser to index each particle in the beam, a pulse ionization laser generator triggered by the timing laser and configured to generate at least one of an IR laser pulse and a UV laser pulse to strike each indexed particle when it reaches an ionization region of the ionization laser to produce at least one of ionized fragments of each indexed particle and photons associated with each indexed particle, a guide tube having an outlet end and disposed between the aerosol beam generator and the ionization region to urge particles to flow nearabout the longitudinal axis of the guide tube, and at least one detector to analyze at least one of ionized fragments and photons associated with each particle and generate unique spectral data associated with each indexed particle. The at least one detector may include at least one of a TOF-MS detector, fluorescence detector, LIBS detector, and a Raman spectrometer. The ionized fragments of each indexed particle and photons associated with each indexed particle may be analyzed using a TOF-MS detector to determine the composition of each particle. In some implementations, the pulse ionization laser may generate IR laser pulse or UV laser pulse when triggered by the continuous timing laser when each particle enters the continuous laser beam. The continuous timing laser generator and the pulse ionization laser generator may be configured to produce the continuous laser beam and the pulse ionization laser beam, respectively, as overlapping beams.

Selecting which indexed particle is to be analyzed may be done by triggering the ionization laser step when at least one property of the indexed particle meets a predetermined threshold value for that property. The composition of the analyzed particle may be determined by generating a plurality of single particle spectra using a TOF-MS detector, aligning each single particle spectra, denoising each aligned single particle spectra, averaging the plurality of aligned and denoised single particle spectra, and comparing the averaged spectra with reference spectra. Aligning single particle spectra may include selecting one or more mass ranges based on a priori information related to the location of mass ranges of interest, selecting one spectrum as a reference spectrum for each mass range wherein a reference spectrum includes a spectrum that is at least one of a preselected spectrum, a spectrum present in a reference data library, and a spectrum developed using the measured single particle spectral data set, and shifting the spectral dataset's peak window to align with the corresponding window in the reference spectrum in the time domain.

Additionally, selecting one spectrum as a reference spectrum developed using a measured data set may include selecting a plurality of measured single particle spectra, calculating the Pearson correlation coefficient (“PCC”) for each spectral data file by cross-correlation with each of the other spectra in the dataset and recording the file's average PCC score, selecting the spectrum with the highest PCC score as the reference spectrum. The aligned single particle spectra may be denoised using single value decomposition techniques (“SVD”). The determining the composition step may further include the steps of at least one of comparing the averaged spectral data with a training spectral data set knowledge base to predict composition, updating the training data set knowledge base, and using machine learning methods to improve the prediction of composition over time. The machine learning methods may include supervised machine learning methods.

In the example aerosol TOF-MS systems disclosed above “indexing each particle” means “time-stamping each particle.” To generate a timing laser to index each particle in the beam” the aerosol TOF-MS system may be configured to index or time-stamp each particle using a timing circuit and counter, for example, as described in, U.S. Pat. No. 5,681,752 and U.S. Pat. Pub. No. 2011/0071764, the disclosures of which are incorporated by reference herein in each of their entireties. Photomultiplier tubes may be used to detect light scattered by a particle when excited by a laser beam. Laser 308 may include one or more of UV laser pulses or IR laser pulses, which may be triggered when each selected indexed particle reaches the ionization region. A single trigger laser (303 or 312) may be used to trigger the ionization laser 308. Laser 308 may be triggered only when at least one of particle size, shape, and fluorescence meets or exceeds a predetermined threshold value for that property. Indexing, besides enabling data fusion of mass spectral data associated with each particle, allows for data fusion of optical properties of each particle.

Additionally, in the example aerosol TOF-MS systems disclosed above, the continuous timing laser generator may be configured to implement additional pre-ionization tasks or features (in addition to triggering) including indexing each particle in the beam, optically characterize particle size, particle shape by polarization, and fluorescence of each indexed particle, and select which indexed particles are to be ionized. The example TOF-MS systems may include a data system to generate a data set that combines optical data with unique mass spectral data, process the combined unique spectral data associated with each indexed particle along with particle size, particle shape by polarization, and fluorescence using data fusion methods to generate compiled spectral data associated with selected indexed particle, and compare with a training data set comprising of a knowledge base of known biological matter spectra to predict composition of the bioaerosol particles.

The supervised learning algorithms as disclosed above require a library of threat signatures and labeled data to learn to classify spectra by agent or hazardous substance. While this approach works well for known threats, methods and systems to detect anomalies and to flag possible unknown agents or next-generation threat agents or hazardous substances are needed. Methods and systems to generate a dataset of mass spectra by in silico mixing mass spectra of known threats and spectra of background are also needed, particularly when it is not possible to release a biological substance into the environment. Application of in silico methods allows measurements of a biological substance under carefully controlled conditions (such as in an aerosol chamber) to be combined with environmental measurements to simulate a predetermined concentration.

### SUMMARY

In some implementations, an example method for predicting an anomaly in a background environment may include the steps of compiling a dataset of reference analyte (hazardous substance) mass spectra of single aerosolized analyte particles representative of hazardous particles present in the environment, compiling a dataset of reference background mass spectra of single aerosolized background environment particles free of any analyte hazardous substance particles, generating a dataset of simulated composite mass spectra of analyte particles in the background environment in silico at different analyte concentrations in the background environment using the reference analyte hazardous substance mass spectra and the reference background mass spectra, identifying an optimum batch size of the composite spectra at each analyte hazardous substance concentration, wherein the batch size represents the number of spectra to be averaged, and determining a threshold error for predicting an anomaly in the single particle mass spectra generated from a new aerosol sample drawn from the environment.

In some implementations, the composite mass spectra at different analyte hazardous substance concentrations may be generated in silico by varying a dilution ratio defined as the number of reference analyte hazardous substance spectra divided by the sum of the number of reference analyte hazardous substance spectra and reference background spectra. The example method may dilute individual analyte hazardous substance particles with individual background particles. In some implementations, the background environment may be ambient air. The analyte hazardous substance particles may include at least one of a chemical substance and a biological substance. The reference analyte hazardous substance spectra and background spectra may be generated using an aerosol MALDI TOF-MS system.

In some implementations, the step of identifying the optimum batch size at each analyte hazardous substance concentration step may include training an autoencoder to learn features characteristic of the reference background spectra, generating Receiver Operator Characteristic curves (“ROC”) based on probability distributions of the autoencoder's reconstruction loss associated with individual hazardous substance spectra and background spectra, and identifying the optimum batch size as the batch size which maximizes area under the ROC curve (“AUC”).

In some implementations, the example method may further include the step of discarding mass spectra features below a predetermined m/z cut-off value prior to the training step. The predetermined m/z cut-off value may be less than about 3000. The determining the threshold error step may include selecting an optimal reconstruction loss threshold from the ROC curve by maximizing Youden's J statistic. The Youden J statistic would give equal weight to sensitivity and specificity, but this is possible when spectra of hazardous substances are available. Also, sensitivity and specificity may have different significance, depending on the use case. For defense applications, high specificity (low false alarms) may be prioritized, even if it results in compromising sensitivity. In some implementations, the optimum batch size may be 1. In some implementations, particles may be tagged to be either anomalous or non-anomalous. Anomalous particles may be subject to further analysis. Non-anomalous particles are discarded.

In some implementations, an example method of diagnosing an anomaly in an environment may include generating single particle mass spectra from aerosol samples collected from the environment continuously or at predetermined intervals, inputting the single particle mass spectra to an autoencoder trained to compress and reconstruct mass spectra based on baseline reference mass spectra of aerosol samples taken from the environment, examining the single particle mass spectra at a predetermined optimal batch size, determining a reconstruction loss threshold associated with the baseline reference mass spectra of aerosol samples taken from the environment, and diagnosing an anomaly in the environment if the reconstruction loss of the autoencoder related to the aerosol samples exceeds the reconstruction loss threshold wherein an anomaly is detected at the level of single particles without implementing batch averaging of mass spectra. The optimum batch size may be 1. The single particle mass spectra from aerosol samples collected from the environment may be generated using an aerosol MALDI TOF-MS system.

In some implementations, an example method for predicting an anomaly in a background environment caused by one or more unknown aerosol analyte hazardous substance particles may include compiling a dataset of reference background mass spectra of single background environment particles, training an autoencoder to learn features characteristic of the reference background mass spectra at varying batch sizes wherein the batch sizes represent the number of mass spectra to be averaged, determining a background mass spectra reconstruction loss threshold at which at least 90% of the background mass spectra are discarded as non-anomalous, and predicting one or more anomalies in single particle mass spectra generated from an aerosol sample drawn from the background environment sample (sample spectra) if the reconstruction loss related to the sample spectra exceeds the reconstruction loss threshold.

In some implementations, the example method may further include the step of discarding mass spectra features below a predetermined m/z cut off value prior to the training step. The predetermined m/z cut-off value may be less than about 3000. Mass spectra may be generated using an aerosol MALDI TOF-MS system. The autoencoder may be trained for a predetermined number of epochs (the number of times that the learning algorithm will work through the entire training dataset) on at least about 20,000 single particle spectra collected from an environment sample. The predetermined number of epochs may be the greater of 20 epochs, or the number of epochs at which the percentage reduction in average training loss is minimized to numerical accuracy, for example, as used in PyTorch or following the IEEE 754 standard. The example method may further include the step of examining an averaged, processed spectrum of sample spectra to determine the presence of one or more features characteristic of an unknown analyte hazardous substance. The example method may further include the step of examining a heat map of processed sample spectra to determine the presence of one or more statistically significant features characteristic of an unknown analyte hazardous substance.

Other features and advantages of the present disclosure will be set forth, in part, in the descriptions which follow and the accompanying drawings, wherein the preferred aspects of the present disclosure are described and shown, and in part, will become apparent to those skilled in the art upon examination of the following detailed description taken in conjunction with the accompanying drawings or may be learned by practice of the present disclosure. The advantages of the present disclosure may be realized and attained by means of the instrumentalities and combinations particularly pointed out in the appendant claims.

### BRIEF DESCRIPTION OF THE DRAWINGS

The foregoing aspects and many of the attendant advantages of this disclosure will become more readily appreciated as the same becomes better understood by reference to the following detailed description, when taken in conjunction with the accompanying drawings, wherein:

FIGS. 1A-B show TOF-MS configurations of (A) linear TOF-MS and (B) combined linear and reflectron TOF-MS.

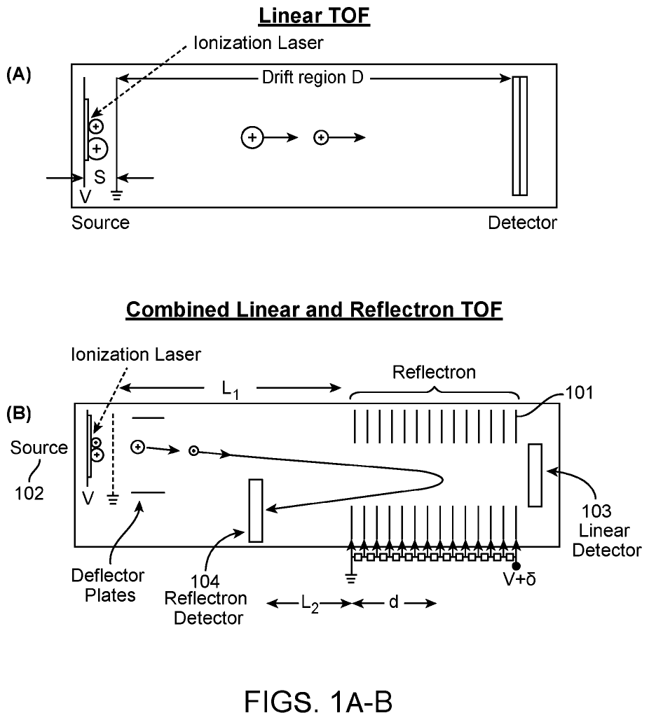

FIG. 2 shows characteristic whole-cell (top-down) MALDI TOF-MS spectrum of Bacillus atrophaeus (Bg) spores acquired on a high-resolution TOF-MS, according to some implementations.

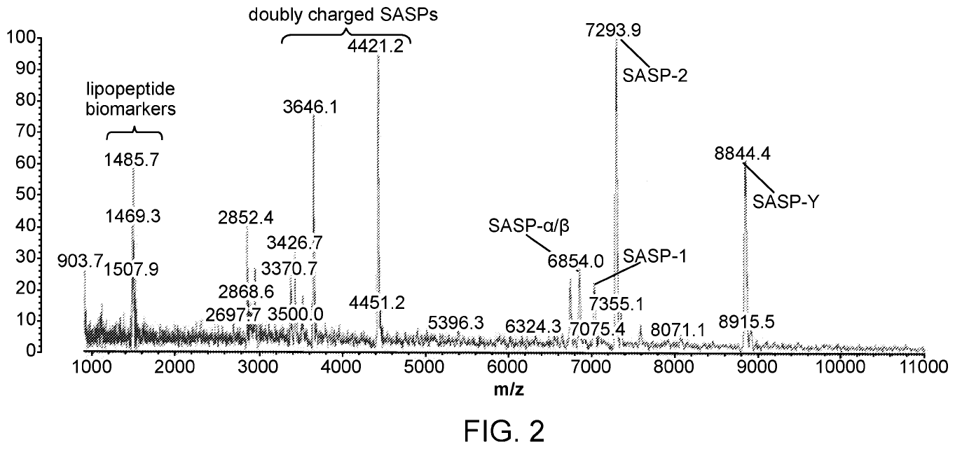

FIG. 3 shows a schematic diagram of an example system for single particle aerosol analysis, according to some implementations.

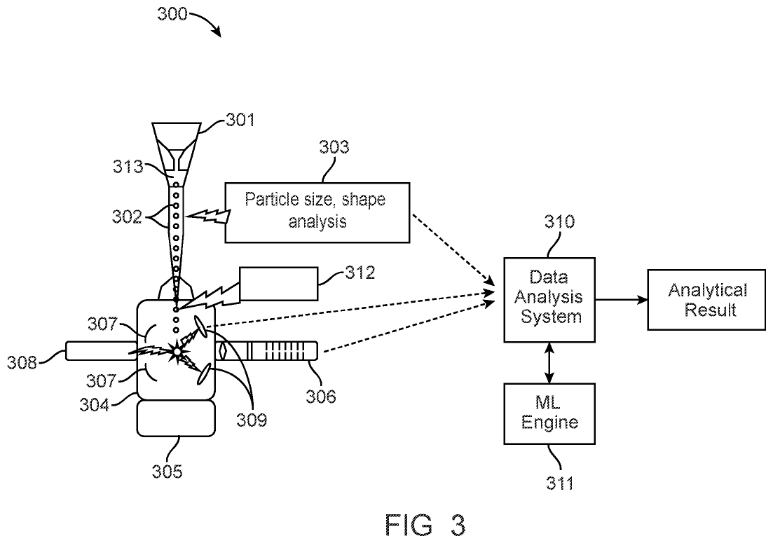

FIGS. 4A-C show heat maps of (A) raw Bg spectra, (B) of background spectra, and of (C) a simulated mixture that randomly draws 10% from the Bg dataset and 90% from the background dataset, according to some implementations.

FIGS. 5A-B show a comparison of simulated and binomial distribution showing the probability distribution of the number of simulant spectra in a (A) batch of 1 and (B) batch size of 5, according to some implementations.

FIG. 6A shows a schematic diagram of a conventional TOF-MS signal processing pipeline.

FIG. 6B shows a schematic diagram of a single particle aerosol MALDI TOF-MS signal processing pipeline, according to some implementations.

FIGS. 7A-C show the effect of signal preprocessing steps in the example signal processing pipeline, according to some implementations.

FIGS. 8A-B show (A) a confusion matrix demonstrating the ability of the KNN classifier to detect and identify live agents against noise, and (B) standard deviation demonstrating higher values for missed samples or misidentified samples, according to some implementations.

FIG. 9 shows a schematic representation of an autoencoder network architecture, according to some implementations.

FIG. 10 shows a schematic diagram of an example method for selecting optimal reconstruction loss threshold for a given threat agent or analyte hazardous substance concentration, according to some implementations.

FIGS. 11A-C show probability distributions of an example autoencoder's reconstruction loss on (A) background vs. simulant Bg spectra at batch size of 1, (B) batch size of 5, and (C) batch size of 10, according to some implementations.

FIGS. 12A-C show Receiver Operator Characteristic (“ROC”) curves based on probability distributions of the autoencoder's reconstruction loss on background vs. simulant Bg spectra and Area under the ROC curve (“AUC”) values for each ROC curve at (A) batch size of 1, (B) batch size of 5, and (C) batch size of 10, according to some implementations.

All reference numerals, designators and callouts in the figures are hereby incorporated by reference as if fully set forth herein. The failure to number an element in a figure is not intended to waive any rights. Unnumbered references may also be identified by alpha characters in the figures and appendices.

The following detailed description includes references to the accompanying drawings, which form a part of the detailed description. The drawings show, by way of illustration, specific embodiments in which the disclosed systems and methods may be practiced. These embodiments, which are to be understood as “examples” or “options,” are described in enough detail to enable those skilled in the art to practice the present invention. The embodiments may be combined, other embodiments may be utilized, or structural or logical changes may be made, without departing from the scope of the invention. The following detailed description is, therefore, not to be taken in a limiting sense and the scope of the invention is defined by the appended claims and their legal equivalents.

In this disclosure, aerosol generally means a suspension of particles dispersed in air or gas. “Real-time” analysis of aerosols generally means analytical methods and devices that identify the aerosol analyte within a matter of minutes after the aerosol sample to be analyzed is introduced to the analytical device or system. The terms “a” or “an” are used to include one or more than one, and the term “or” is used to refer to a nonexclusive “or” unless otherwise indicated. In addition, it is to be understood that the phraseology or terminology employed herein, and not otherwise defined, is for the purpose of description only and not of limitation. Unless otherwise specified in this disclosure, for construing the scope of the term “about,” the error bounds associated with the values (dimensions, operating conditions etc.) disclosed is ±10% of the values indicated in this disclosure. The error bounds associated with the values disclosed as percentages is ±1% of the percentages indicated. The word “substantially” used before a specific word includes the meanings “considerable in extent to that which is specified,” and “largely but not wholly that which is specified.”

### DETAILED DESCRIPTION

Particular aspects of the invention are described below in considerable detail for the purpose of illustrating the compositions and principles, and operations of the disclosed methods and systems. However, various modifications may be made, and the scope of the invention is not limited to the example aspects described.

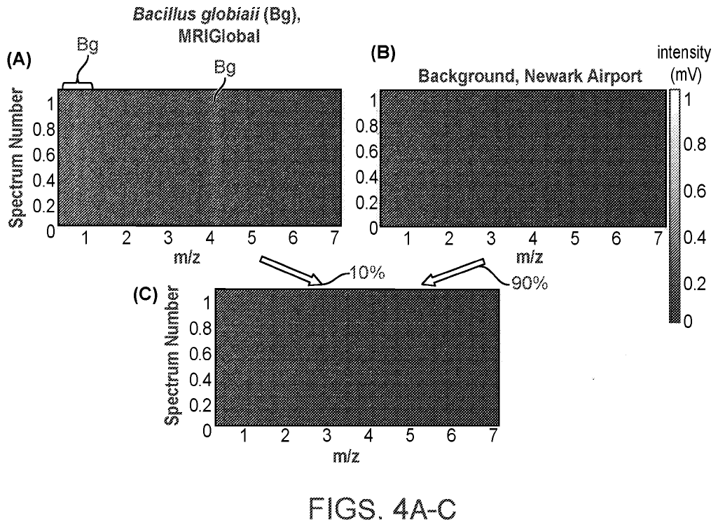

To better understand the detection capabilities of a threat agent or hazardous substance in, for example, ambient air and using aerosol TOF-MS, it is important to assess both the true positive rate and false positive rate at a wide range of concentrations. Since it is difficult to methodically prepare agent-containing-aerosol concentrations, an example anomaly detection method may include diluting individual simulant (threat agent or analyte hazardous substance) particles with individual background particles at a specified ratio in silico (computationally or using computer simulation). The simulation accepts a dataset of background spectra, a dataset of simulant spectra, a desired dilution (as a percentage), and an output dataset size, and randomly samples from the two datasets accordingly. As described below in Example 1 (FIGS. 4A-C), the heatmaps illustrate that even a 10× dilution can profoundly reduce the ability to detect a threat agent or analyte hazardous substance with the human eye. While averaging spectra in batches (compared to single particle spectra or a single average) can enhance the separation of signal from noise, the optimal batch size is dependent on the agent or analyte hazardous substance concentration. At sufficiently low dilutions, it may be undesirable to perform any batch averaging.

For instance, if there were only one threat spectrum and four background spectra in a batch of five spectra, averaging all five together would only decrease the signal-to-noise ratio. However, if four out of five spectra contained threat agent or analyte hazardous substance mass spectra features, averaging all five spectra would improve the signal-to-noise ratio. For ambient air analysis, it is important to test different dilution ratios since that represents different threat concentrations in air. The dilution ratio may be defined as m/(n+m), where n refers to normal instances (normal background), and m to anomalies. The example data curation method disclosed herein for environmental monitoring for biothreats in single particle mass spectra analysis computationally dilutes individual simulant particles with individual background particles at a specified ratio. The example method circumvents the need to methodically scale agent-containing-aerosol concentrations in the laboratory at different dilution ratios. The simulation accepts a dataset of background spectra, a dataset of simulant (threat agent or analyte hazardous substance) spectra, a desired dilution (as a percentage), and an output dataset size, and randomly samples from the two datasets accordingly. The example method permits the study of cases of a defined, limited number of threat particles in an arbitrary background over a period of operation.

The probability P (x) of “drawing” either a threat containing spectrum or a background spectrum (specific outcome) within n trials from a random sample (of the environment) of spectra may be represented by the binomial distribution:

$$P ( x ) = ( \begin{matrix}n \\ x\end{matrix} ) {p}^{x} {q}^{n - x} = \frac{n !}{( n - x ) ! x !} {p}^{x} {q}^{n - x}$$

where n is the number of trials or iterations sampled at a time (the batch size), x is the number of simulants per sampling, p is the probability of drawing a simulant set in one iteration or single trial (probability of success), q=1−p is the probability of drawing from background set in one iteration (probability of failure) on a single trial.

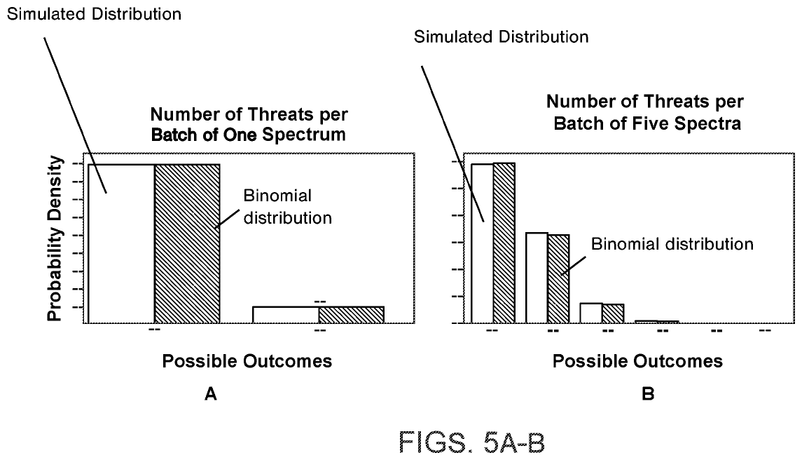

FIGS. 5A-B show a comparison of simulated and binomial distribution showing the probability distribution of the number of simulant spectra in a (A) batch of 1 and (B) batch size of 5, according to some implementations. As can be seen, there is very close agreement between the simulated distribution and binomial distribution for batch size of 1 and 5, with p=10%. Accordingly, the anomaly detection methods disclosed herein offer a data curation process that may, in principle, be applied to any detection system, and is of particular interest in the realm of aerosol applications such as aerosol TOF-MS, where airborne agent or analyte hazardous substance concentrations are not well defined.

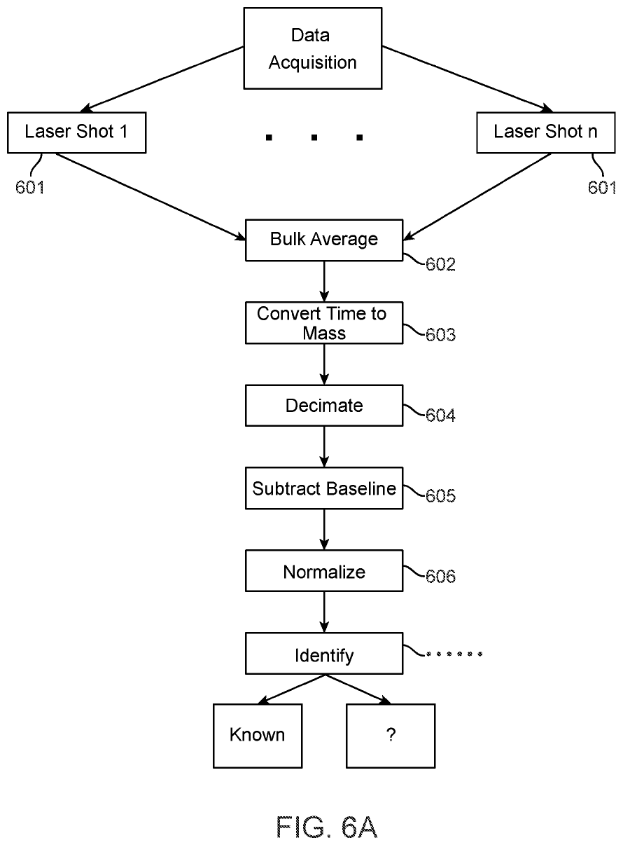

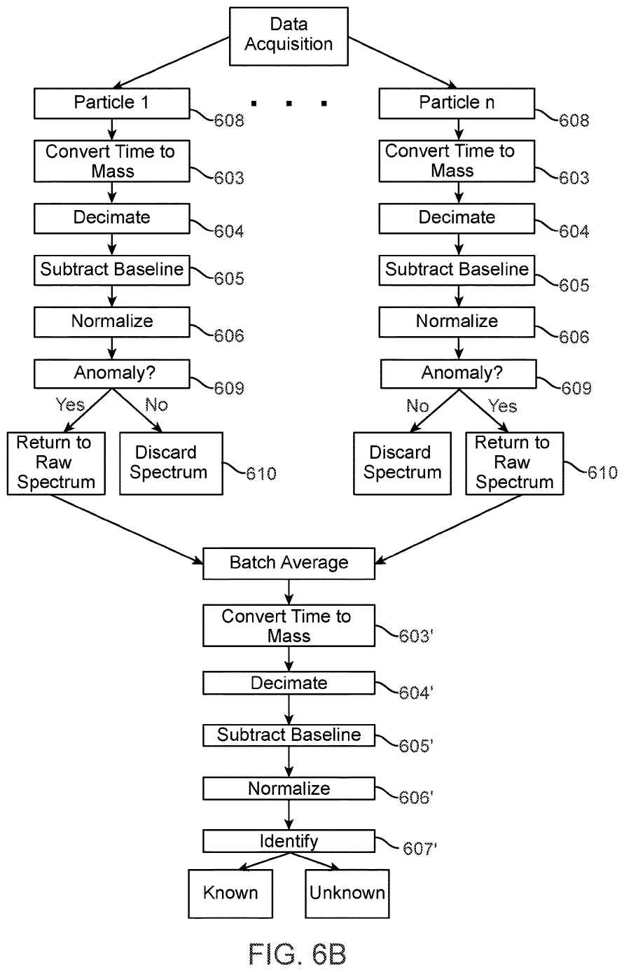

In conventional MALDI TOF-MS analysis, a bulk sample is co-deposited on a plate with matrix, placed under vacuum in the mass spectrometer, and desorbed and ionized by a laser. FIG. 6A shows a schematic diagram of a conventional TOF-MS signal processing pipeline. A laser is fired repeatedly (typically on the order of hundreds of times) while the beam position is randomly scanned over the spatial distribution of the sample. Each laser shot produces a mass spectrum in step 601, and the recorded spectra are averaged together at once in step 602 prior to further signal preprocessing (steps 603-606) and identification. Converting flight times of charged particles to mass in step 603 was previously described. Decimation in step 604 is the process of reducing the sampling rate. In practice, this usually implies lowpass-filtering a signal, which reduces the number of data points in a spectrum, thereby helping to manage computational requirements like memory and processing speed.

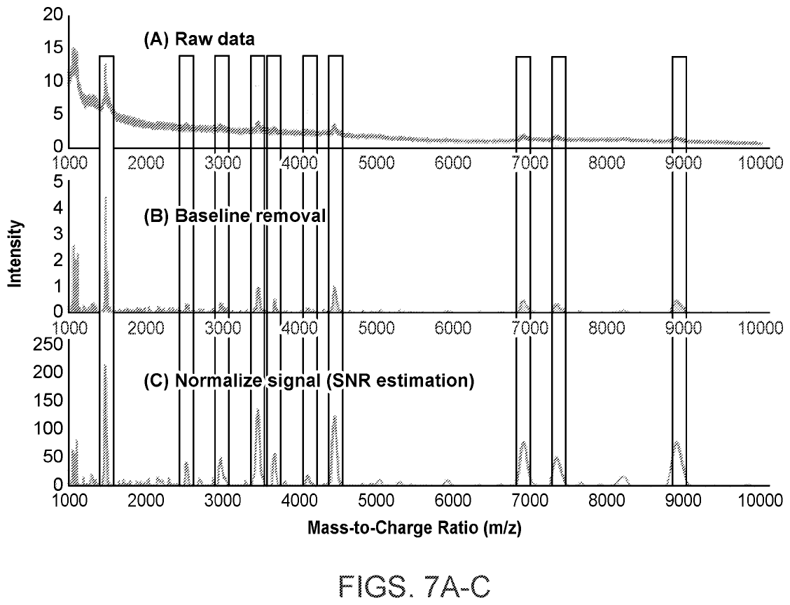

FIGS. 7A-C show the effect of signal preprocessing steps in the example signal processing pipeline, according to some implementations. FIGS. 7A-C illustrate the impact of baseline subtraction (and filtering and smoothing) and normalization (steps 605-606) preprocessing steps on the quality of the signal. The baseline drift (FIG. 7A) that produces a very high signal level at low masses and decays exponentially with increasing m/z is due to ions from the matrix combined with other fragments of the sample. A median filter may be used to estimate and subsequently remove this baseline. A popular noise reduction technique, the median filter, is computationally efficient, effective in non-gaussian noise distributions, and has minimal impact on the information containing peaks associated with the agent or analyte hazardous substance signature (FIG. 7B). Normalization step 606 may estimate the signal to noise ratio (“SNR”) at each m/z value. This normalization process greatly enhances the SNR of the signature, especially for higher masses (FIG. 7C).

As m/z increases, the variation of the noise decreases considerably, and small variations in the signal become statistically significant. While this example method shown in FIG. 6A works reasonably well in a laboratory with high-grade scientific equipment and offline sample preparation techniques (e.g., centrifugation, lyophilization, culturing, etc.), indiscriminately averaging spectra produced by all laser shots together can introduce unnecessary chemical noise that will obscure weak signals. The detection of weak signals necessitates more detailed spectral information and informed signal processing. Identification step 607 may include feature extraction and identification. The process is optimized to preserve discrete spectral peaks while reducing broader non-stationary noise features.

FIG. 6B shows a schematic diagram of a single particle aerosol MALDI TOF-MS signal processing pipeline, according to some implementations. In single particle aerosol MALDI TOF-MS, mass spectra of individual airborne particles are acquired in step 608 and processed using steps 603-606 as previously disclosed. In a cluttered environment such as ambient air, threat agents or analyte hazardous substances are highly diluted, and a majority of these single particles are bound to be characteristic of the background. For this reason, preliminary preprocessing of each particle spectrum generated from step 608 is performed in steps 603-606, and spectra of particles are determined to be anomalous (spectrum has features not representative of background spectra) or not in step 609. Those spectra that are determined to be anomalous are flagged for further analysis, and the rest are discarded in step 610.

At steps 603′-606′, the raw anomalous spectra may be processed and identified in batches. The process shown in FIG. 6B differs from the conventional MALDI processing scheme shown in FIG. 6A in that an anomaly detector in step 609 is applied as a filter to individual particle spectra. In addition to improving the sensitivity to known threats, this approach may also flag the presence of unknown threat agents or analyte hazardous substance because the anomaly detection step 609 only looks for spectra with features not representative of background. Even after the TOF-MS spectra preprocessing methods as described above, MALDI-TOF mass spectra are inherently complex data representations of biological and chemical markers present in a sample, and therefore require significant expertise to be properly interpreted by a human.

Typically, spectra are classified using a supervised machine learning algorithm trained on a library of well-documented agent or analyte hazardous substance signatures, but this approach neglects to identify unknown signatures. It is critical to be prepared for unknown, next-generation threats, and the use of an unsupervised anomaly detection model is required. Therefore, an anomaly detector at 609 may be employed for single particle data analysis to eliminate single particle mass spectra that do not appear to contain a biological agent signature from a large volume of mass spectra of aerosolized particles, for example, when the particles are collected from ambient air in federal office building, airports or from other critical threat areas. With potentially thousands of spectra per second to process, human analysis is impractically resource-intensive and prone to bias.

Machine learning may be used to classify large and diverse sets of data rapidly and accurately. In machine learning, a mathematical model may be trained on a set of data to make predictions based on new data. These models may range in complexity from simple statistical regressions to artificial neural networks. Data processing steps such as feature selection from mass spectra and identification may be implemented with machine learning algorithms. Machine learning techniques require input mass spectral features to be fed to the model. Feature extraction of MALDI mass spectral data associates sets of peaks with particular biological agents or analyte hazardous substances. Because the location of peaks is known a priori, this knowledge may be used to significantly reduce the dimensionality (reduce the number of significant mass spectra features) of the mass spectra data. Machine learning methods may provide information related to which features are used in classification, unlike those discovered by unsupervised clustering methods such as Principal Component Analysis (“PCA”). Background samples (for example, ambient air with no biological or chemical threat agents or analyte hazardous substance) are assumed to have no mass spectra features. After salient features are extracted for a wide range of biological or chemical agents or analyte hazardous substances, a k-nearest neighbors (“KNN”) algorithm may be used to classify each sample as either background or agent (analyte hazardous substance).

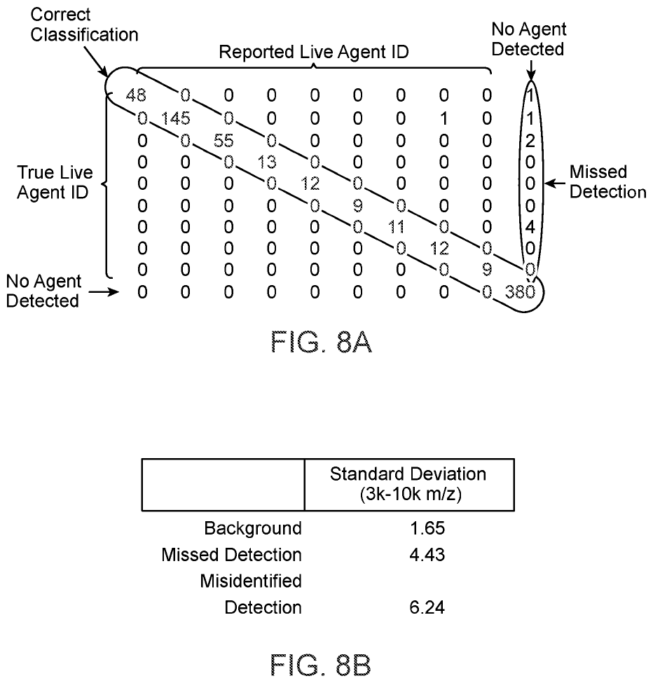

A KNN algorithm takes an unlabeled test vector and classifies it by assigning the label that is most frequent among the k training samples closest to that test vector, where closeness is in terms of distance between vectors in the multidimensional feature space. FIGS. 8A-B show (A) a confusion matrix demonstrating the ability of the KNN classifier to detect and identify live agents against noise, and (B) standard deviation demonstrating higher values for missed samples or misidentified samples, according to some implementations. The KNN classifier as described below in Example 2, and FIGS. 8A-B, is attractive for mass spectra data analysis as it makes no assumptions about the distribution of the data, which is an advantage particularly when the dataset is small (for example, less than about 1000, but the size of the data set may vary). If the number of features is also small (for example, less than about 50, but may vary), it is easy to add new training data without significant processing resources. The limited number of features may likely exclude meaningful information contained in less prominent peaks, but the large reduction in dimensionality lessens the likelihood of overfitting, which is a common problem when a model is useful only in reference to its training dataset and does not generalize to other data.

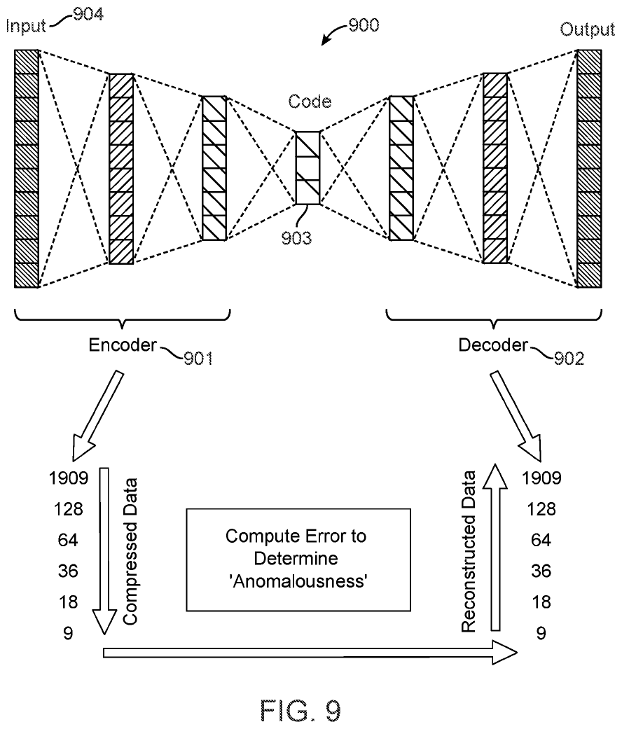

Example methods for a generalized anomaly detection may include (a) estimating distribution parameters, such as the mean and variance of certain features in a peak window or across the entire spectrum, and (b) extracting background features with deep learning, such as autoencoders. The distribution parameters may relate to a quantity that may be computed for each spectrum and characterized by some difference between threat and background spectra. For example, the variance of a spectrum might be higher if there are some biological/chemical particles present. FIG. 9 shows a schematic representation of an autoencoder network architecture, according to some implementations. An autoencoder 900 is an unsupervised deep learning method composed of two symmetrical neural networks, namely, an encoder 901 and a decoder 902. These neural networks may be made of linear or convolutional layers. The encoder compresses an input data vector to a lower-dimensional space (“code” 903); that is, it compresses the input data to only the most critical features, which include the critical intensities in mass spectra. Decoder 902 may use a similar structure to reconstruct the input vector from its compressed data representation. In doing so, the autoencoder learns the most efficient representation of the input data because features are not defined as important and not important features a priori in the input data.

Further, spectra are not classified as background spectra or anomalous threat spectra in the input data. Input vector 904 may include a vector of the intensities of a mass spectrum, with a lower m/z cutoff of 3,000 to eliminate the chemical noise associated with a MALDI matrix. Example autoencoder 900 may be trained for about 20 epochs (the number of times that the learning algorithm will work through the entire training dataset) on about 20,000 single particle spectra collected, for ambient air, and averaged, for example, in batches of five, to smooth any noise spikes. The number of epochs is determined by examining whether there is any gain from additional training. When the improvement in the model is marginal, the training process may be stopped.

In autoencoder 900, the model architecture goes from 1909 down to 9 features and consists of a linear encoder and decoder, with each linear layer followed by a rectified liner unit (“ReLU”) activation function. The final step of the decoder is a Sigmoid activation function that outputs values between 0 and 1. The network may be built using the open-source machine learning library PyTorch. Sequentially, the encoder begins with a linear layer going from 1909 to 128 features, then a ReLU activation function, then a linear layer from 128 to 64, then another activation function, then another linear layer from 64 to 36, and so on until 9 is the final dimension. Then the decoder scales back up to 1909.

During the training step, the model may compress an example input vector from 1909 features to 9 features and learn to reconstruct the output vector to be as similar as possible to the input vector, while minimizing reconstruction error. When a test vector is compressed and expanded, the error between the input and the output indicates how similar the test vector is to the training data. The autoencoder will generally reproduce a normal trace input with minimal error, but the abnormal trace input will exhibit a significant difference between the input and output traces. The autoencoder may seek to minimize the loss function, which may be defined as the mean squared error (“squared L2-norm”) between the target value and the estimated value. Therefore, the reconstruction loss acts as an anomaly score that can be thresholded to eliminate normal signals and flag anomalous signals potentially from threat agents or analyte hazardous substance for further analysis.

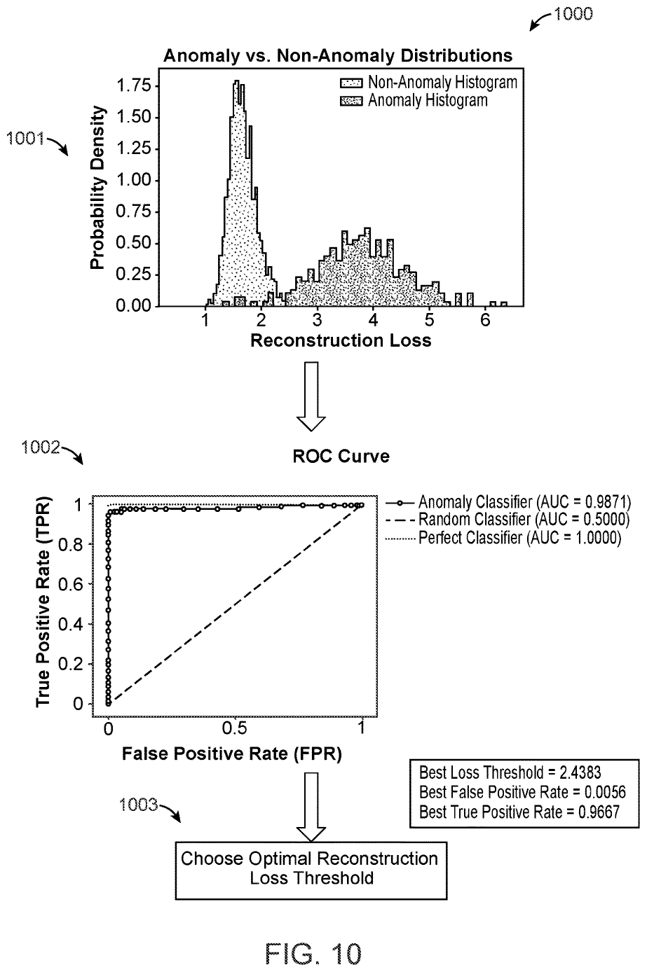

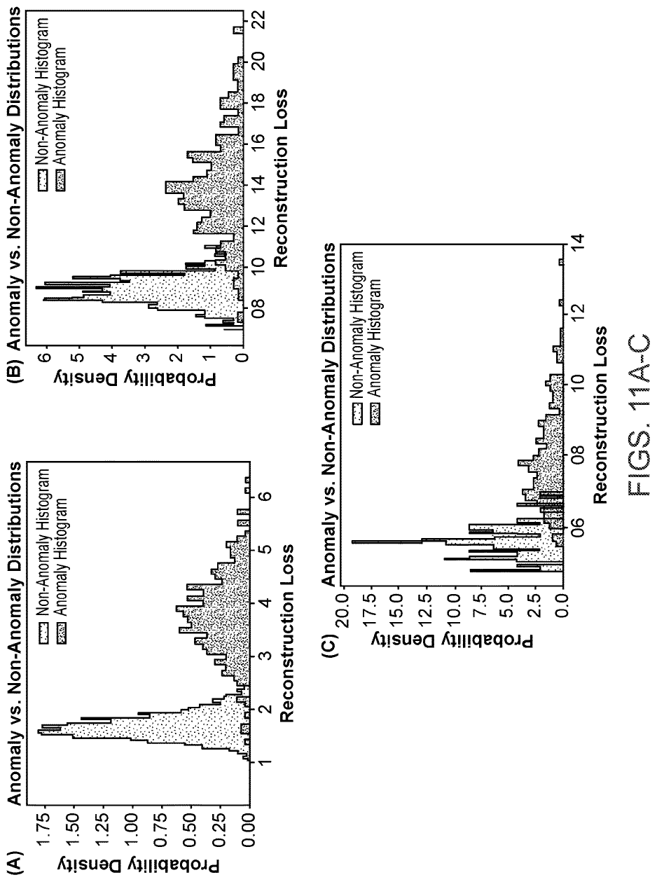

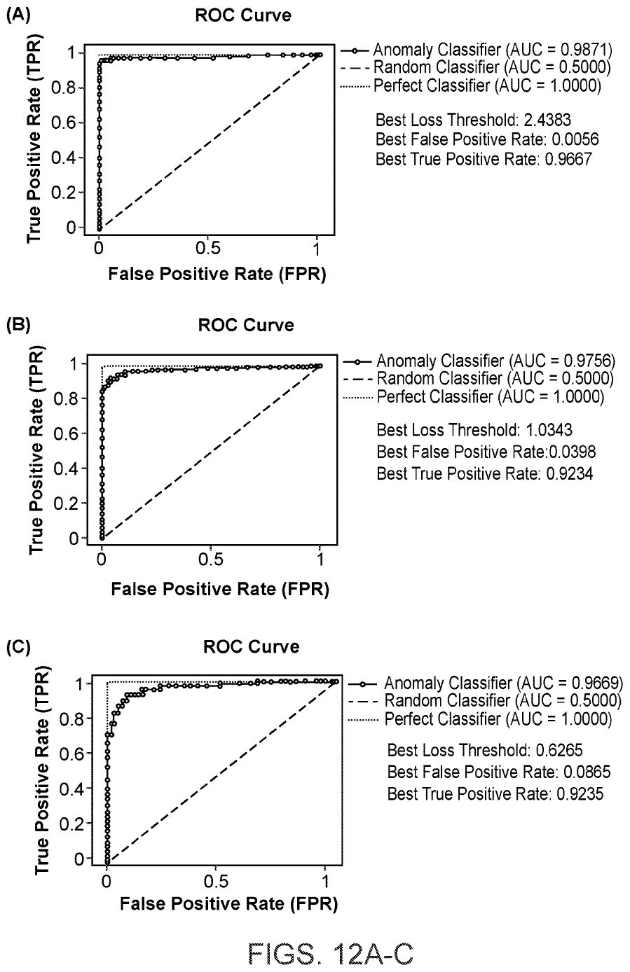

FIG. 10 shows a schematic diagram of an example method 1000 for selecting an optimum reconstruction loss threshold for a given threat agent or analyte hazardous substance concentration, according to some implementations. Example method may include the following steps:
(a) In step 1001, generating Receiver Operator Characteristic curves (“ROC”) based on probability distributions of the autoencoder's reconstruction loss on background vs. simulant (threat) spectra. For example, FIGS. 11A-C show probability distributions of an example autoencoder's reconstruction loss on (A) background vs. simulant Bg spectra at batch size of 1, (B) batch size of 5, and (C) batch size of 10, according to some implementations.
(b) In step 1002, select which best batch size provides a maximizing area under the ROC curve (“AUC”) while comparing batch sizes at the same threat agent or analyte hazardous substance concentration in the aerosol. In FIG. 10, the best batch size is 1. For example, FIGS. 12A-C show Receiver Operator Characteristic (“ROC”) curves based on probability distributions of the autoencoder's reconstruction loss on background vs. simulant Bg spectra and Area under the ROC curve (“AUC”) values for each ROC curve at (A) batch size of 1, (B) batch size of 5, and (C) batch size of 10, according to some implementations.
(c) In step 1003, select an optimal reconstruction loss threshold from the ROC curve by maximizing Youden's J statistic, defined as (sensitivity+specificity−1). This parameter gives equal weight to sensitivity and specificity. Specificity may be defined as (TN/TN+FP) and sensitivity as (TP/TP+FN) where TP, FP, TN, FN refer to true positive, false positive, true negative, and false negative outcomes, respectively.

Finally, the model may be tested by compressing and expanding a test data vector and by examining the error between the input and the output, which would indicate how similar the test data vector is to the training data. Therefore, an autoencoder may be trained on environmental background data to reduce the spectra to a few important features and to determine if a test spectrum deviates from that of the background rather than to reduce the size of the spectra dataset. The spectra that the autoencoder determines are similar to the background spectra are discarded, thereby reducing the number of spectra to be analyzed further (as previously described in steps 609-610 referring to FIG. 6B). Based on the autoencoder's ability to identify anomalies, the example methods show that for exceedingly sparse signals, such as signals of threat agents or analyte hazardous substance in ambient air, it is best to examine single spectra rather than a bulk average of spectra, as is normally done.

As can be clearly seen in FIGS. 12A-C, anomalies were detected at the level of single particles without any batch averaging of mass spectra. Further, autoencoder performance was best at the single particle level (batch size of 1, FIG. 12A) with a high anomaly classifier AUC value of 0.9871, a true positive rate of 0.9667, and very low false positive rate of 0.0056. This was unexpected because single spectra are generally considered to be too noisy to be classified (background vs threat) well by specific algorithms. As a result, there is no need to average spectra in batches (compared to single particle spectra or a single average) to enhance the separation of signal from noise. It appears that single spectra may be too noisy to be classified at the level of strain/organism, but not too noisy to be classified at the level of background vs. anomaly. In the anomaly detection process, no a priori assumptions are made as to the composition and type of the threat agent or analyte hazardous substance, the autoencoder is trained to learn single particle (individual aerosol particle) mass spectra features of the environment (background) using a large training data set, and if a measurement deviates from the background, it is flagged for further analysis.

The example methods disclosed herein were implemented using a deep learning workstation (Lambda Labs) having the following example specifications:
(a) Operating system, Ubuntu 20.04, includes Lambda Stack for managing TensorFlow, PyTorch, CUDA, cuDNN, and others,
(b) Processor: AMD Threadripper 3960X: 24 cores, 3.80 GHz, 128 MB cache, PCIe 4.0,
(c) CPU Cooler: air Cooling,
(d) GPUs: 1×RTX A4000, 16 GB,
(e) Memory: 32 GB (2×16 GB, 3200 MHz, and
(f) Operating system drive: 1 TB SSD (NVMe).

The code was written in the web-based Jupyter interface that runs Python and uses many of its open-source libraries, for example, PyTorch for machine learning, Pandas for data manipulation and analysis, Plotly for making interactive graphs, and the like.

### EXAMPLES

### Example 1. Simulating Diluted Concentrations of Airborne Threats

Using a single particle MALDI-TOF mass spectrometer, two datasets were acquired of raw Bacillus globigii (Bg) spectra (MRI Global) and of raw background spectra of ambient air samples obtained at Newark Airport, to simulate a challenge of 3,000 threat agent or analyte hazardous substance particles at a dilution of 10%. In other words, 10% of these 3,000 spectra were simulants (threat agents or analyte hazardous substance) and the remaining 90% were background. FIGS. 4A-C show heat maps of (A) raw Bg spectra, (B) of background spectra, and of (C) a simulated mixture that randomly draws 10% from the Bg dataset and 90% from the background dataset, according to some implementations. The heatmaps in FIG. 4A-C illustrate that even a 10× dilution can profoundly reduce the ability to detect a threat with the human eye. Additionally, as shown in FIG. 5A-B, using the numbers from the aerosol simulation as described above (p=300/3000=0.1, q=1−p=0.9), close agreement between the theoretical binomial distribution and the empirical simulated distribution for batch sizes (n) of one (single particles) and five, suggest that the probability distribution of the number of simulant spectra in a batch of a given size is binomial. This exercise demonstrates the distribution of limited threat particles in an arbitrary background over a period of operation. Further, the impact of processing spectra in batches of different sizes and at different concentrations may be explored.

### Example 2. Classification of Aerosol TOF-MS Mass Spectra Features of Ambient Air Samples Using a KNN Classifier

FIGS. 8A-B show (A) a confusion matrix demonstrating the ability of the KNN classifier to detect and identify live agents against noise, and (B) standard deviation demonstrating higher values for missed samples or misidentified samples, according to some implementations. The KNN classifier was used to classify aerosol TOF-MS mass spectra of over 2000 ambient air samples including background samples (with no biological agents) and samples including biological agents or analyte hazardous substances. The agents included multiple spore strains, vegetative bacteria, virus, and toxin with a wide range of concentrations. A confusion matrix generated using live agent or analyte hazardous substance data and a subset of the background samples is shown in FIG. 8A. No false detections were observed. Given the wide range of challenge concentrations, some missed detections (agent or analyte hazardous substance identified as background) and misidentified detections (agent or analyte hazardous substance incorrectly identified) of weak signals were observed. These misclassifications were caused by weak responses of the selected features combined with an overall increase in the noise level in the spectra. While this was not unexpected, it is important to note that the system was able to detect the presence of agents or analyte hazardous substance with low response to specific features. FIG. 8B illustrates that a simple measure of the signal standard deviation in the 3,000-10,000 m/z range correlates well with the presence of an agent or analyte hazardous substance, as the standard deviation of missed detections and misidentified detections was significantly higher than that measured for background alone. The distribution of intensities in these background spectra was found to be stable. As a result, machine learning methods may be used to extract features of the background, and then for any sample, determine if it is consistent with the background distribution.

The example systems and methods disclosed herein may be used for a wide range of applications including, but not limited to, critical infrastructure protection to clinical diagnostics. In the biodefense arena, deployment in transit systems, sports and entertainment venues, or government facilities has the potential to detect the intentional release of biological warfare agents or biological analyte hazardous substance. In the field of medicine, they can be used to rapidly analyze exhaled breath for disease-causing pathogens, either for individual screenings at entry points or early point-of-care diagnosis of respiratory infection.

The Abstract is provided to comply with 37 C.F.R. § 1.72 (b), to allow the reader to determine quickly from a cursory inspection the nature and gist of the technical disclosure. It should not be used to interpret or limit the scope or meaning of the claims.

Although the present disclosure has been described in connection with the preferred form of practicing it, those of ordinary skill in the art will understand that many modifications can be made thereto without departing from the spirit of the present disclosure. Accordingly, it is not intended that the scope of the disclosure in any way be limited by the above description.

It should also be understood that a variety of changes may be made without departing from the essence of the disclosure. Such changes are also implicitly included in the description. They still fall within the scope of this disclosure. It should be understood that this disclosure is intended to yield a patent covering numerous aspects of the disclosure both independently and as an overall system and in both method and apparatus modes.

Further, each of the various elements of the disclosure and claims may also be achieved in a variety of manners. This disclosure should be understood to encompass each such variation, be it a variation of an implementation of any apparatus implementation, a method or process implementation, or even merely a variation of any element of these.

Particularly, it should be understood that the words for each element may be expressed by equivalent apparatus terms or method terms-even if only the function or result is the same. Such equivalent, broader, or even more generic terms should be considered to be encompassed in the description of each element or action. Such terms can be substituted where desired to make explicit the implicitly broad coverage to which this disclosure is entitled. It should be understood that all actions may be expressed as a means for taking that action or as an element which causes that action. Similarly, each physical element disclosed should be understood to encompass a disclosure of the action which that physical element facilitates.

In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Further, the use of the transitional phrase “comprising” is used to maintain the “open-end” claims herein, according to traditional claim interpretation. Thus, unless the context requires otherwise, it should be understood that variations such as “comprises” or “comprising,” are intended to imply the inclusion of a stated element or step or group of elements or steps, but not the exclusion of any other element or step or group of elements or steps. Such terms should be interpreted in their most expansive forms so as to afford the applicant the broadest coverage legally permissible.

1. (canceled)

2. The method of claim 21, wherein the simulated composite mass spectra representative of single analyte hazardous substance particles in the background environment at different analyte hazardous substance concentrations is generated in silico by varying a dilution ratio defined as the number of reference analyte hazardous substance mass spectra divided by the sum of the number of reference analyte hazardous substance mass spectra and the number of reference background mass spectra.

3. The method of claim 21, wherein the background environment is ambient air.

4. The method of claim 21, wherein the analyte hazardous substance particles comprise at least one of a chemical substance and a biological substance.

5. The method of claim 21, wherein the reference analyte hazardous substance mass spectra and the reference background mass spectra are generated using an aerosol MALDI TOF-MS system.

6. The method of claim 21, wherein identifying the optimum batch size of the simulated composite mass spectra at each analyte hazardous substance concentration comprises:
training an autoencoder to learn features characteristic of the reference background mass spectra;
generating Receiver Operator Characteristic curves (ROC) based on probability distributions of the autoencoder's reconstruction loss associated with the analyte hazardous substance mass spectra and the background mass spectra; and
identifying the optimum batch size as the batch size which maximizes area under the ROC curve (AUC).

7. The method of claim 6, further comprising discarding mass spectra features below a predetermined mass-to-charge ratio (m/z) cut-off value prior to the training step.

8. The method of claim 7, wherein the predetermined m/z cut-off value is less than about 3000.

9. The method of claim 6, wherein determining the threshold error comprises selecting an optimal reconstruction loss threshold from the ROC curve by maximizing Youden's J statistic.

10. The method of claim 21, wherein the optimum batch size is 1.

11. A method of detecting an anomaly in an environment, the method comprising:
generating single particle mass spectra from aerosol samples collected from the environment continuously or at predetermined intervals;
inputting the single particle mass spectra to an autoencoder trained to compress and reconstruct mass spectra based on baseline reference mass spectra of aerosol samples taken from the environment;
examining the single particle mass spectra at a predetermined optimum batch size, wherein a batch size represents the number of single particle mass spectra to be averaged;
determining a reconstruction loss threshold associated with the baseline reference mass spectra of aerosol samples taken from the environment; and
detecting an anomaly in the environment if a reconstruction loss of the autoencoder associated with the single particle mass spectra of the aerosol samples exceeds the reconstruction loss threshold.

12. The method of claim 11, wherein the predetermined optimum batch size is 1.

13. The method of claim 11, wherein the single particle mass spectra from aerosol samples collected from the environment are generated using an aerosol MALDI TOF-MS system.

14. A method for predicting an anomaly in a background environment caused by one or more unknown aerosol analyte hazardous substance particles, the method comprising:
compiling a dataset of reference background mass spectra of single background environment particles;
training an autoencoder to learn features characteristic of the reference background mass spectra at different batch sizes, wherein a batch size represents the number of reference background mass spectra to be averaged;
determining a background mass spectra reconstruction loss threshold at which at least 90% of the background mass spectra are discarded as non-anomalous;
generating sample mass spectra including single particle mass spectra generated from an aerosol sample drawn from the background environment; and
predicting an anomaly in the background environment if the reconstruction loss related to the sample mass spectra exceeds the background mass spectra reconstruction loss threshold.

15. The method of claim 14, further comprising discarding mass spectra features associated with the reference background mass spectra below a predetermined m/z cut-off value prior to the training step.

16. The method of claim 15, wherein the predetermined m/z cut-off value is less than about 3000.

17. The method of claim 14, wherein the sample mass spectra are generated using an aerosol MALDI TOF-MS system.

18. The method of claim 14, wherein the autoencoder is trained for a predetermined number of epochs on at least about 20,000 single particle mass spectra associated with the sample mass spectra.

19. The method of claim 18, wherein the predetermined number of epochs is the greater of 20 epochs or the number of epochs at which a percentage reduction in average training loss is minimized to numerical accuracy as defined under the IEEE 754 standard.

20. The method of claim 14, further comprising the step of examining a heat map of processed sample spectra to determine the presence of one or more statistically significant features characteristic of an unknown aerosol analyte hazardous substance.

21. A method for predicting an anomaly in a background environment, the method comprising:
generating a dataset of simulated composite mass spectra representative of single analyte hazardous substance particles in the background environment in silico at different analyte hazardous substance concentrations in the background environment using reference analyte hazardous substance mass spectra and reference background mass spectra;
identifying an optimum batch size associated with the simulated composite mass spectra at each analyte hazardous substance concentration, wherein a batch size represents the number of simulated composite mass spectra to be averaged; and
determining a threshold error for predicting an anomaly in a single particle mass spectra generated from a new aerosol sample drawn from the environment.

22. The method of claim 21, wherein the reference analyte hazardous substance mass spectra includes mass spectra associated with single aerosolized analyte hazardous substance particles representative of hazardous particles present in the background environment.

23. The method of claim 21, wherein the reference background mass spectra includes mass spectra associated with single aerosolized background environment particles free of any analyte hazardous substance particles.
